(19) United States
(12) Patent Application Publication
(10) Pub. No.: US 2002/0058699 A1
(43) Pub. Date: May 16, 2002

(54) Methods for treating seizure disorders by inhibiting MAPK pathway activation

(76) Inventors: J. David Sweatt, Houston, TX (US); Anne E. Anderson, Houston, TX (US)

Correspondence Address: Stephen A. Bent, FOLEY & LARDNER, Washington Harbour, 3000 K Street, N.W., Suite 500, Washington, DC, 20007-5109, US

(21) Appl. No.: 09/893,754

(22) Filed: Jun. 29, 2001

Related U.S. Application Data
(60) Provisional application No. 60/215,221, filed on Jun. 30, 2000.

(51) Int. Cl. A61K 31/277

(52) U.S. Cl. 514/523

(57) ABSTRACT

Excessive brain neuronal excitability, associated with a seizure disorder, can be correlated with increased mitogen-activated protein kinase (MAPK) activity in neurons. Such excessive excitability can be ameliorated by administering an effective amount of a compound, such as a MAPK phosphorylation or kinase activity inhibitor, that reduces the amount of MAPK activity in neurons of an individual suffering from a seizure disorder. Compounds that inhibit phosphorylation or kinase activity of upstream activators or downstream targets of the MAPK cascade also are useful in this context.

### STATEMENT REGARDING FEDERALLY SPONSORED RESEARCH

The invention was made in part with government support under grant nos. NS37444 and NS01836 awarded by the National Institutes of Neurological Disorders and Stroke. Accordingly, the government may have certain rights in the invention.

### FIELD OF THE INVENTION

The present invention relates generally to the use of inhibitors of Mitogen-Activated Protein Kinase (MAPK) to modulate the activity of potassium (K⁺) channels which affect neuronal excitability. More specifically, the invention relates to the use of MAPK inhibitors for the treatment of epilepsy and other disorders involving dysfunctional neuronal MAPK activity.

### BACKGROUND OF THE INVENTION

A seizure represents a discrete, abnormal episode of hyperexcitability in brain structures, influencing motor or sensory function, behavior, or consciousness. Seizures can occur in a variety of situations including epilepsy, Parkinson's disease, trauma, drug withdrawal, tetanus, metabolic disease, elevated body temperature, drug induction (e.g., with theophylline) or in developmental disorders including autism, cerebral palsy, and inborn errors of metabolism.

Epilepsy is a clinical paroxysmal disorder of recurring seizures, excluding alcohol or drug withdrawal seizures or such recurring exogenous events as repeated insulin-induced hypoglycemia. Epilepsy includes disorders of recurring seizures resulting from a brain tumor or stroke.

About 10 percent of all Americans will have at least one seizure at some time. Many people have one or a few attacks and then never have another one. Epilepsy with recurrent seizure is significant, affecting 0.5-1% of the population involving individuals of all ages.

Drug therapy is the most widespread treatment of epilepsy. Presently known anti-epileptic drugs such as phenytoin, carbamazepine, valproic acid or phenobarbital generally are believed to prevent or control seizures by acting on pathologically altered neurons or normal cells having restricted vascular supply or an injured area in which the neurons of a nerve net have been destroyed. Such drugs act to temporarily reduce and control the epileptic seizures as opposed to curing the underlying disorder.

For those with recurrent seizures, about 70 percent are reasonably controlled with anti-epileptic drugs. However, anti-epileptic drugs have a significant toxicity profile including induction of cerebellar-vestibular effects, skin disorders, hematological disorders, hepatic deficiencies and congenital abnormalities. Furthermore, of the 150,000 people who develop epilepsy each year, 10 to 20 percent have a “medically intractable epilepsy,” not responsive to drug therapy.

Several approaches are proposed for treating medically intractable epilepsy. These include neurosurgery or the application of electromagnetic materials and electromagnetic fields generated for example by lasers, microwaves and radio frequency. However, surgery has risks and costs that must be considered including the certainty of the diagnosis and the adequacy of previous drug therapy while electromagnetic materials and electromagnetic fields are not currently medically accepted.

### SUMMARY OF THE INVENTION

It therefore is an object of the present invention to provide a more effective approach for treating disorders resulting from excessive neuronal excitability such as epilepsy. It is also an object of the present invention to identify more specific targets for treating disorders based on excessive neuronal excitability such as epilepsy by administering compounds that effect the functioning of such targets.

In accomplishing these and other objectives, there has been provided, in accordance with one aspect of the present invention, a method of reducing or inhibiting excessive neuronal excitability in an individual resulting from an increased mitogen-activated protein kinase (MAPK) activity in neurons of the individual, comprising administering an effective amount of a compound that reduces the amount of MAPK activity in the neurons.

There also is provided, in accordance with another aspect of the present invention, a method of reducing or inhibiting excessive neuronal excitability in an individual resulting from an increased mitogen-activated protein kinase (MAPK) activity in neurons of the individual, comprising administering an effective amount of a compound that inhibits phosphorylation or kinase activity of the MAPK cascade downstream of MAPK.

In a further aspect of the present invention, a method is provided for reducing or inhibiting seizures in an individual suffering from a seizure disorder, resulting from increased mitogen-activated protein kinase (MAPK) activity, comprising administering an effective amount of a compound that inhibits phosphorylation of potassium channels.

### BRIEF DESCRIPTION OF THE DRAWINGS

FIG. 1 is an observation graph demonstrating the kinetics of development of seizure activity in male rats administered 15 mg/kg kainate IP versus a control group (saline).

FIG. 2 is an observation graph demonstrating that SL327 pretreatment blocks limbic motor status epilepticus in kainate-treated rats. SL327 (400 mg/kg) or vehicle (PEG 400/ethanol) was administered intraperitoneally (IP), 30 minutes prior to the administration of 15 mg/kg kainate (KA; see arrows).

FIG. 3 demonstrates steady-state activation curves for control and PD098059 (an MEK inhibitor) showing a hyperpolarizing shift in the voltage-dependence of K⁺currents recorded in dendrites. MEK inhibitor PD098059 (50 M) or vehicle was applied to the hippocampal CA1 slices at least 20 minutes before recording conductance. Conductance with inhibitor was increased compared to controls at almost all voltages applied, resulting in a phase shift of the inhibitor to the left of the control. Inset graph demonstrates the conduction currents when PD 098059 was washed in, after recording a control activation curve. Control traces show less transient current at all potentials compared in the physiologic range to traces with the PD compound wash.

### DETAILED DESCRIPTION OF THE PREFERRED EMBODIMENTS

The present inventors have discovered that excessive neuronal excitability such as typifies epilepsy results from an increased kinase activity of mitogen-activated protein kinase (MAPK) in brain neurons. Such excessive neuronal excitability can result in a seizure disorder. The increased neuronal activity stems from an increased phosphorylation of MAPK in brain neurons, particularly in neurons of the hippocampus.

The increased MAPK activity in brain neurons involves the MAPK subgroup of extracellular signal-regulated kinases (ERKs). ERKs are proline-directed protein kinases which phosphorylate Ser/Thr-Pro motifs. ERK1 and ERK2 are abundantly expressed in the mature central nervous system (CNS). Boulton et al., Cell 65:663-675 (1991). Phosphorylated, activated ERK2 is more highly expressed in the hippocampus and appears to be localized to the soma and dendrites of hippocampal pyramidal neurons. Fiore et al., Neuroscience 55:463-472 (1993).

It has been determined that increased MAPK kinase activity in neurons causing excessive neuronal excitability including seizures stems from increased phosphorylation of the brain MAPK isoforms ERK1 (44 Kd or p44) and ERK2 (42 Kd or p42), respectively. The increased kinase activity of ERKs is largely due to dual phosphorylation of tyrosine and threonine motifs, although some activation may result from single-site phosphorylation. A singly phosphorylated ERK has about a 5-10 fold increase in kinase activity while a dually phosphorylated ERK has about a 3,000 fold increase in kinase activity. Anderson et al., Nature 343:651-653 (1990); Cobb et al., Prog. Biophys. & MoL Biol. 71:479-500 (1999).

Based on these discoveries, it is apparent that the amount of activated MAPK in brain neurons plays a central role in acute and chronic stages of disorders based on excessive neuronal excitability seizure such as epilepsy. The present invention addresses these problems by reducing or inhibiting by reducing the amount of MAPK activity in a neuron. “Excessive” means a level of excitability that is statistically greater than the level of excitability observed in normal individuals. Excessive neuronal excitability may be characterized by an increased intensity or duration of the neuronal signaling.

Accordingly, the present invention contemplates reducing or inhibiting excessive neuronal excitability including seizures in an individual suffering from an increased neuronal MAPK activity. This is accomplished by administering an effective amount of a compound that reduces the amount of MAPK activity in the neuron. “Reducing or inhibiting” means decreasing the severity and/or the frequency of the seizures.

Excessive neuronal excitability is present in a variety of neural seizure disorders including, for example, epilepsy, Parkinson's disease, trauma, drug withdrawal, tetanus, metabolic disease, elevated body temperature, or drug induction (e.g., theophylline). Reducing or inhibiting excessive neuronal excitability through MAPK can help to down-regulate gene transcription which leads to chronic changes, such as hippocampal cell loss and circuitry changes (i.e., mossy fiber sprouting), that are associated with epilepsy. MAPK also can regulate gene expression directly through regulation of transcription factor activation and this process can be inhibited using MAPK pathway inhibitors as described herein. Seizures that are associated with epilepsy are a preferred target for an application of the present invention.

Reducing the amount of MAPK activity in a cell can be accomplished by a variety of approaches. One approach is dephosphorylation of the mono- or di-phosphorylated form of MAPK that exists in a neuron. Compounds that activate mono (Ser/Thr or Tyr) or dual-specific threonine/tyrosine phosphatases, present in neurons, are useful for this purpose. Ward et al., Nature 367:651-654 (1994); Chu et al., J. Biol. Chem. 271:6497-6591 (1996).

Approaches for reducing the activity of MAPK also include methods for inhibiting MAPK phosphorylation. This can be achieved by reducing the phosphorylation state of upstream members of the MAPK cascade. Because the phosphorylation state of upstream members of the MAPK cascade are linked to members downstream, methods that reduce the activation state of members upstream of MAPK ultimately affect the activation state of MAPK.

In the context of this description, the phrase “MAPK cascade” denotes the signal transduction pathway that exists upstream of MAPK and that results in activation of MAPK, typically by single or dual phosphorylation. The MAPK cascade can be initiated with the activation of tyrosine kinase receptors located at the cellular plasma membrane by a wide variety of growth factors including various neurotrophins. Seeger et al., Proc. Natl. Acad. Sci. (USA) 88:6142-6146 (1991); Cobb Prog. Biophys. & Mol. Biol. 71:479-500 (1999). In addition, MAPK can be activated by various cell-surface receptors coupled to second messenger cascades. These plasma membrane receptors include the N-methyl-D-aspartate (NMDA) glutamate receptor subtype as well as -adrenergic, muscarinic, mGluR, and dopaminergic receptors, all of which are coupled in the cascade to MAPK located in the hippocampus. Roberson et al., J. Neurosci. 19:4337-4348 (1999). Activated tyrosine kinase receptors deliver signals down the cascade by activating Ras, which in turn activates Raf to then phosphorylate and activate MAP kinase/ERK kinase (MEK), a dual specificity (serine/threonine) protein kinase. Crews et al., Science 258:478-480 (1992); Zheng et al., J. Biol. Chem. 268:23933-23939 (1993). Protein kinase C (PKC) and Protein kinase A (PKA) also lead to activation of kinases upstream of MEK in CA1 hippocampal neurons. Roberson et al, J. Neurosci. 19:4337-4348 (1999). PKA and PKC activation appears to be selective for but not specific for p42 MAPK (ERK2). An additional route of MAPK activation is by ligand-gated ion channels that depolarize the membrane and/or allow calcium influx such as glutamate or acetylcholine receptors.

Compounds that act at the activation step nearest in the pathway to MAPK should generally be more effective in reducing the activation state of MAPK. Inhibiting the ability of a MEK, which is a pathway member immediately upstream of MAPK, to phosphorylate MAPK is a preferred approach for reducing the activity of MAPK.

There are three known isoforms of MEK, identified as MEK1, MEK2 and MEK3. These isoforms phosphorylate and fully activate p44 (ERK1) and p42 (ERK2) MAPK, by dual phosphorylation of threonine and tyrosine residues. MEK1 is highly expressed in the adult CNS and appears to be the primary regulator of MAPK activation in mature neurons. Crews et al., Science 258:478-480 (1992). Accordingly, compounds that inhibit MEK1 are preferred for the present invention (see Example 3, below). Compounds that inhibit the activity of other upstream kinases in the MAPK cascade such as Ras, Raf1, B-Raf and Rap1 also are useful to reduce MAPK activity as are inhibitors of adaptor/scaffolding proteins such as shc, sos and grb.

The amount of activated MAPK in a neuron can be reduced by approaches that cause dephosphorylation of upstream kinases in the MAPK cascade. Thus, compounds that activate phosphatases specific for any members of the MAPK cascade upstream of MAPK will reduce the activity of the upstream kinase, ultimately leading to reduced downstream activity of MAPK. Compounds that effect dephosphorylation of MEK are preferred because this kinase is directly upstream of MAPK in the cascade. However, compounds that effect dephosphorylation of other upstream kinases including Ras, Raf1, B-Raf and Rap1 also can be used.

Another approach to reduce the amount of MAPK activity in a neuron involves reducing the amount of MAPK protein or upstream kinase that is present in a neuron. This approach becomes effective when a particular cascade member is present at concentrations that become rate-limiting in the cascade. Down-regulating the amount of a particular protein in a cell can be accomplished by administering antisense compounds or by other genetic manipulations well known in the art. Antisense therapy involves the administration of exogenous oligonucleotides that bind to a target nucleic acid, typically an RNA molecule located within cells. The term “antisense” refers to the fact that the oligonucleotides typically are complementary to at least a portion of the target nucleic acid (e.g., mRNA) or the “sense strand” which encodes the cellular product to be down-regulated. The ability to use anti-sense oligonucleotides to inhibit expression of mRNAs, and thereby to inhibit protein expression in vivo, is well documented, for example, in U.S. Pat. No. 5,948,680. Approaches to selecting an appropriate antisense sequence for down-regulating a particular gene also have been described, for example, in U.S. Pat. No. 6,060,248.

In other approaches, down-regulation of a protein can be achieved by, for example, oligonucleotide compositions with high affinity for a target transcription factor which can be introduced into cells as decoy cis-elements to bind the factor and alter gene expression (see U.S. Pat. No. 6,060,310). Alternatively, chemicals that alter the expression of genes within the cell can be used for gene down-regulation. These must be specifically directed to interact with the regulatory components of a particular gene of interest to avoid general down-regulation of genes and cell toxicity.

Yet another approach to reducing the activity of MAPK entails inhibiting the ability of MAPK to phosphorylate downstream targets of the MAPK cascade, including signal-transduction members between MAPK and the final end products activated by phosphorylation. In this regard, compounds are preferred that directly block the ability of MAPK to phosphorylate a downstream effector, including a transcriptional factor such as elk-1 (Impey et al., Neuron. 21:869-83 (1998); Roberson et al., J. Neurosci. 19:4337-4348 (1999); Adams et al., Soc. Neurosci. Abstr. 23:1176 (1997)), ribosomal S6 kinase 2 (RSK2) and its target CREB, or potassium channel subunits such as Kv4.2. The present description of the “MAPK cascade,” as that phrase is used here, is not intended to encompass the stress-induced protein kinase pathway or calcium/calmodulin-activated kinase pathways.

Those of skill in the art can readily identify compounds suitable for reducing the amount of MAPK activity in a neuron, as described above. Presently preferred compounds include the vinylogous cyanamides, represented below by formula 3 (Example 6), including Z- and E-α-(amino ((4-aminophenyl) thio)methylene)-2-(trifluoromethyl) benzeacetonitrile, referred to herein as SL327 (“compound 16” in Example 6), U0126 [Duncia et al., Bioorg. Med. Chem. Lett. 8:2839-2844 (1988); or PD98059[2-(2′-amino-3′-methoxyphenyl)-oxanaphthalen-4-one; Dudley et al., Proc. Natl. Acad. Sci. (USA) 92:7686-7689 (1995); Weiss et al., Am. J. Physiol. 274:C1521-1529 (1988)], which inhibit the kinase activity of the MEK (also known as MAPK kinase). Vinylogous cyanamides such as SL327 are particularly attractive because they can be administered in a routine manner (e.g., subcutaneously or intravenously) and can cross the blood-brain barrier. The synthesis of vinylogous cyanamide class of MAPK inhibitors including SL327 is described in Example 6.

Other useful compounds that inhibit the activity of kinases upstream of MAPK in the MAPK cascade include, for example, cyclic AMP-dependent kinase inhibitors, H7, staurosporine, genistein (5,7-dihydroxy-3-(4-hydroxyphenyl)-4H-1-benzopyran-4-one), and the c-Raf inhibitor, SP 203580 and the like. Compounds that act on RAS and inhibit activation of the ERK cascade include farnesyl transferase inhibitors such as B-581 [Garcia et al., J. Biol. Chem. 268:18415-18418 (1993)] BZA-5B [Dalton et al., Cancer Res. 55:3295-3304 (1995)], farnesyl acetate, and (x-hydroxyfarnesyl) phosphonic acid [Gibbs et al., J. Biol. Chem. 268:7617-7620 (1993)].

Many compounds described above are widely available commercially. For example, genistein is available from LC Laboratories (Woburn, Mass.). Those compounds that are not commercially available can be readily prepared using organic synthesis methods known in the art.

Compounds for achieving a reduction in phosphorylation of MAPK in brain neurons also can be screened from candidate compounds, using techniques described here. Thus, Examples 1-5 relate methodology for identifying whether a compound is an inhibitor of MAPK activation or an inhibitor of potassium channel Kv4.2 activation, and, if so, whether the compound is effective in reducing or eliminating abnormal neuronal membrane excitability or in reducing or eliminating seizures, during acute and chronic phases of epileptogenesis in an animal model.

The amount of MAPK activity in a neuron is reduced by administering an effective amount of a compound with properties as discussed above. An “effective amount” is the amount of the compound that significantly reduces MAPK activity, which can be measured by detecting the amount of preferably dually phosphorylated MAPK present in the neurons. Phosphorylated MAPK can be readily detected using specific antibodies that are commercially available, such as from New England Biolabs (Beverly, Mass.) or Upstate Biotechnology (Lake Placid, N.Y.), or are readily prepared by methods well known in the art, as illustrated below in Examples 4 and 5. Other established methods for detecting activated MAPK include the “gel shifting” assay (English and Sweatt, J. Biol. Chem. 271:24329-24332 (1996)) and MAPK immunoprecipitation followed by measurement of substrate phosphorylation.

A dose range that achieves an effective amount of the compound described herein for use in humans can be estimated from cell culture assays and animal studies by standard pharmaceutical procedures. For example, the median lethal dose (LD₅₀) is the dose lethal to 50% of an experimental animal population and the median effective dose (ED₅₀) is the therapeutically effective dose in 50% of the population. The ratio of the LD₅₀ to the ED₅₀ is a measure of drug safety, known as the therapeutic index. Compounds which exhibit large therapeutic indices are preferred. A preferred dosage in vivo typically falls within a range of systemic drug concentration that achieves an ED₅₀ with little to no toxicity. The dosage may vary within this range depending upon the dosage form employed and the route of administration utilized. Generally, a therapeutically effective amount is achieved by administering between about 0.1 to about 50 mg/kg body weight, depending on a number of factors including, for example, its EC₅₀ IC₅₀ and on the age, size and condition of the patient.

Compounds for use in the methods herein can be administered to an individual, either alone or as a component in a pharmaceutical formulation, where the compound is mixed with suitable carriers or excipient(s). The exact formulation, route of administration, and dosage are chosen using conventional methodology. For example, see Fingl et al., in THE PHARMACOLOGICAL BASIS OF THERAPEUTICS (1975), Chapter 1. Techniques are well-known for formulating a compound with pharmaceutically acceptable carriers, suitable for a desired route of administration. For instance, see REMINGTON'S PHARMACEUTICAL SCIENCES, 18th ed. (Mack Publishing Co., 1990). The compounds may be formulated as tablets, pills, capsules, liquids, gels, syrups, slurries, suspensions and the like, for oral ingestion by a patient to be treated if feasible. Alternatively, the compound may be encapsulated into liposomes.

Suitable routes for administering the compounds may include direct brain instillation, oral, rectal, transdermal, vaginal, transmucosal, or intestinal administration; parenteral delivery, including intramuscular, subcutaneous, intramedullary injections, as well as direct intraventricular, intravenous, intraperitoneal, intranasal, or intraocular injections, just to name a few. Formulations are preferred that assist the crossing of the blood brain barrier.

The present invention is described, in further detail, by reference to the following examples, which are illustrative only.

### EXAMPLE 1

### Detection of MAPK Activation in Hippocampus, After Kainate-Induced Seizures

This example demonstrates a method to detect activation of MAPK in the hippocampus of animals following the development of seizures induced by kainic acid. The kainate rat model was chosen for these studies because it mimics human epilepsy. Acutely following the administration of kainic acid, rats develop continuous seizures (status epilepticus) involving the limbic structures. Chronically, these animals exhibit spontaneous seizures and hippocampal sclerosis with neuronal loss in hilar, CA1, and CA3 brain regions and sprouting of the granule-cell mossy fibers in the inner molecular layer (mossy fiber sprouting).

The kainate model of epilepsy closely resembles one of the most common epilepsies in humans, temporal lobe epilepsy. The seizures associated with temporal lobe epilepsy, complex partial seizures, involve the limbic structures and are often refractory to medical management. The most common pathological finding in temporal lobe epilepsy is hippocampal sclerosis.

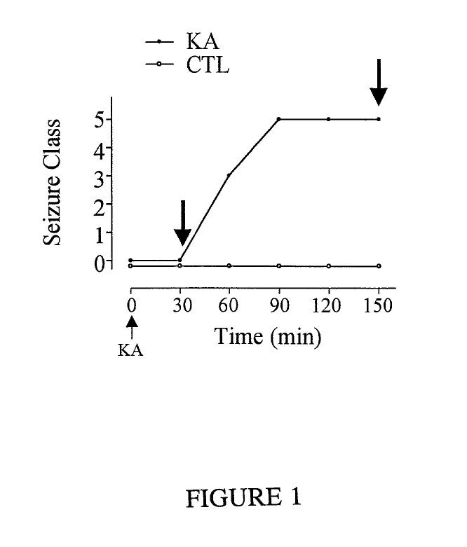

Male Sprague-Dawley rats (125-200 gm) were administered kainate at a dose of 15 mg/kg IP while the control animals (CTL) received vehicle IP (saline). Seizures were scored independently by two investigators in accordance with the Racine Classification Scale (0=no seizures, 1=mouth/face movements; 2=head nodding; 3=forelimb clonus; 4=rearing; and 5=rearing and falling). [Ben-Ari et al., Devl. Brain. Res. 14:284-288 (1985)]. Seizure score is shown plotted against time in the kainate-treated and control (vehicle-injected) animals. None of the kainate-treated animals exhibited behavioral seizures during the first 30 min following kainate treatment, while all of the animals studied developed class 5 seizures within 90 min after kainate that persisted until the 150 min time point (FIG. 1).

Animals were sacrificed 30 min after kainate administration (prior to the behavioral seizures) and after 1 hr of continuous limbic motor seizures (class 5 seizures) following anesthetization with ketamine/xylazine. Vehicle-treated (control) animals were evaluated at the same time points.

Hippocampal ERK activation was quantified by Western blotting using the method described by Atkins el al.[Atkins et al., Nat. Neurosci. 1:602-609 (1998)] with minor modifications. In brief, animals were sacrificed at 30 minutes and after 1 hr of class 5 seizures (150 min post injection) and the hippocampus was dissected. CA1, CA3 and dentate subregions were dissected in ice-cold saline and homogenates prepared by extracting with denaturing detergent. Protein concentrations were deteremined by Bradford Assay. Homogenates were subjected to Western blotting (immunoblots) prepared by routine methods (for example, see Harlow and Lane, in ANTIBODIES: A LABORATORY MANUAL, Cold Spring Harbor Laboratory Press (1988)). Blotted protein was detected using antibodies to total ERK (1:10,000) and dually phosphorylated, activated ERK MAPK (p42 and p44)(1:1000)(Cell Signaling). Enzyme activity associated with a secondary antibody was detected by incubation with Enhanced Chemiluminescent Reagent (BioRad Laboratories, Hercules Calif.) as substrate. Immunoreactivity of the total ERK and dually phosphorylated activated ERK MAPK in the blot was quantified using densitometry (Scion) for representative blots of p42 and analyzed by Student's t-test.

ERK2 activation was evaluated because synaptic plasticity studies have shown that the greatest change in activation of ERK MAPK is in ERK2 compared to ERK1 isoform in hippocampus. Densitometry showed that at the early time point (30 min after kainate), prior to behavioral seizures, there was a significant increase in phospho-ERK2 immunoreactivity in the kainate group compared to controls [KA: CA1 218.9 (±40.66), CA3 206.80 (±31.61), dentate 305.50 (±58.26)% CTL (n=6)]. The significant increase in phospho-ERK2 immunoreactivity in hippocampal subfields in the kainate-treated animals compared to controls persisted at the later time point (150 min) when class 5 seizures were evident (CA1 165.5±16.5%, CA3 153.1±10.3%, and dentate 224.8±34.1% of control)(KA n=7; CTL n=9). Student's t-test, * p<0.05, **p<0.01, ***p<0.001. No difference was seen with blots developed using the total MAPK antibody.

Because no overt behavioral signs of seizures are typically detectable in the KA model at 30 minutes after kainate administration, it can be concluded that MAPK activation precedes the development of status epilepticus. Thus, MAPK activation occurs prior to onset of overt behavioral seizures resulting from kainate-induced status epilepticus, and MAPK activation continues during the seizure period.

Immunohistochemical analysis of stained neural tissue was performed as described by Varga et al., Learn Mem 7;321-322 (2000), with the following modifications. In this case, kainate and control animals were sacrificed at 150 minutes following injection, while in stage 5 seizures for about 1 hour. After tissue fixation, 20-micron sections were thaw-mounted and incubated in 0.3% H₂O₂ in methanol for 30 minutes at RT, washed in PBST (PBS containing 0.2% Triton X-100), and blocked in PBST containing 5% normal goat serum (NGS). Sections were then incubated with the phospho-ERK antibody (1:100) overnight, washed with PBST, and incubated with a 1:200 dilution of goat anti-rabbit biotinylated secondary antibody for 30 minutes at RT. After washing and incubation with a 1:50 dilution of ABC reagent (Pierce) for 30 minutes at RT, sections were again washed in PBST and then stained using metal enhanced DAB. Sections were then rinsed, cleared, and coverslipped using a xylene based medium.

The results of immunohistochemistry confirmed the Western blotting results and demonstrated pronounced hippocampal ERK activation. Specific changes in discrete synaptic zones following seizures were observed. Phospho-ERK staining in stratum oriens of CA1, stratum radiatum of CA1 and CA3, the dentate hilus, and the mossy fiber inputs to CA3 increased greatly following kainate-induced seizures. Although somewhat less dramatic, there also was an increase in detectable phospho-ERK in the inner molecular layer of the dentate following seizures.

The Western blotting and immunohistochemistry showed that kainate seizures are associated with selectively localized changes in hippocampal ERK activation.

### EXAMPLE 2

### Use of MAPK Inhibitors to Inhibit Activation of Hippocampal MAPK in Viva

This example demonstrates that the MEK inhibitor, SL327, is effective in inhibiting the basal level of hippocampal MAPK activation in vivo following IP injection of the drug. This method is useful for selecting therapeutic candidate inhibitors of MAPK phosphorylation that inhibit hippocampal MAPK activation in vivo.

SL327 is a non-competitive inhibitor of MAPK activation that crosses the blood brain barrier. Thus, SL327 can be injected intraperitoneally and will reach the brain. The chemical formula of SL327 is shown as compound 16 in Example 6, which details its method of synthesis. This compound is actually a mixture of the Z- and E- isomers of compound 16.

Adult rats were injected IP with SL327 100 mg/kg or vehicle alone (PEG400/ETOH) were administered 30 minutes later with kainate or vehicle (saline). Animals were sacrificed at following 1 hour of kainate-induced seizures or three hours after kainate injection. Hippocampal regions CA1, CA3, and dentate were removed and processed for Western blotting, as described in Example 1.

SL327 at 100 mg/kg did not block kainate-induced seizures as 100% of these animals had class 5 seizures. No obvious lethargy or other behavioral changes was observed in control animals treated with SL327 100 mg/kg. Furthermore, 100 mg/kg of SL327 did not block kainate-induced ERK activation as seen by Western blotting, while the basal level of ERK activation in control animals was reduced [control+SL327 100 mg/kg: CA1 46.05 (±15.89), CA3 37.51 (±9.59), dentate 32.16 (±11.46)% control (p<0.001); kainate+vehicle: CA1 215.40 (±24.19), CA3 196.60 (±6.69), dentate 219.60 (±8.82)% control (p<0.001); kainate+SL327 100 mg/kg: CA1 220.70 (±35.80), CA3 143.60 (±22.26), dentate 195.6 (±18.58)% control (p<0.001); KA+Vehicle compared to KA+SL327 100 mg/kg p=ns].

### EXAMPLE 3

### Reduction of Kainate-induced Seizures by Inhibiting MAPK Activation

This example shows that high dose SL327 administered prior to treatment with kainic acid reduces or eliminates the generation of limbic motor seizures.

A. High-dose SL327 Pretreatment Blocks Onset of Kainate-induced Seizures

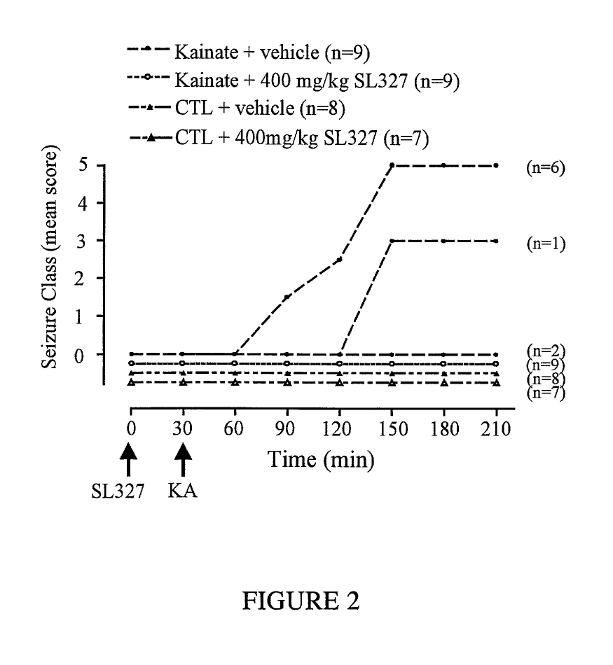

Animals were treated as described in Example 2 except that SL327 was administered at 400 mg/kg. Pretreatment with SL327 at this dose effectively blocked kainate-induced limbic motor seizures (FIG. 2). In contrast, the majority of animals in the vehicle-treated kainate group (no SL327) (78%) developed limbic motor seizures: 5 with class 5 and 1 with class 3 seizures; 6/9 animals had progressed to Class 5 seizures (continuous limbic motor seizures with rearing and falling), while 1/9 had progressed to Class 3 seizures (forelimb clonus), and 2/9 showed no evidence of motor seizures (FIG. 2). A group of animals treated with kainate and no vehicle (n=7, not shown) demonstrated no discernable difference in seizure manifestation or seizure frequency compared to the kainate+vehicle group.

Animals from all groups that were treated with SL327 400 mg/kg exhibited a modest degree of apparent sedation following administration of the MEK inhibitor. Some animals in all SL327 groups (kainate and control) demonstrated an increase in locomotor activity characterized by walking, circling, and random limb movements, but in no case were behaviors characteristic of limbic seizures observed.

Thus, MAPK activation is necessary for the expression of kainate-induced limbic motor seizures and seizures can be blocked by reducing the amount of activated MAPK present in brain neurons.

B. High-dose SL327 Pretreatment Increases MEK Brain Activity

ERK phosphorylation in brain tissue was evaluated in kainate challenged animals pretreated with 400 mg/kg SL327. MEK CA1, CA3, and dentate were dissected and processed for Western blotting as described in Example 1. Representative blots probed with the antibody to dually phosphorylated, activated ERK (phospho-ERK) demonstrated that increased ERK phosphorylation that follows kainate-induced seizures did not arise in animals pretreated with 400 mg/kg SL327. Treatment with SL327 significantly attenuated basal (CTL+SL327 400 mg/kg) and kainate-induced (KA+SL327 400 mg/kg) ERK phosphorylation [CTL+SL327: CA1 8.4±7.2, CA3 2.9±0.5, and dentate 3.1±0.8% of CTL+Vehicle and KA+SL327: CA1 31.5±10.5, CA3 26.2±10.8, and dentate 24.7±9.9% of CTL+Vehicle (KA+SL327 n=9, CTL+SL327 n=7)]. Tukey's Multiple Comparison Test: ***p<0.001.

Blots probed with the antibody against total ERK1/ERK2 MAPK showed no changes in total protein.

### Example 4

### Hippocampal, Shal-type K⁺Channel, Kv4.2, as a Downstream Target of the MAPK- Cascade in Seizure Disorders

This example shows that MAPK regulates the activity of Shal-type K⁺channel, Kv4.2, in the hippocampus of animals with kainate-induced status epilepticus, indicating that voltage-dependent K channel, Kv4.2 and MAPK are targets for controlling seizure development in epilepsy. This example also details a method for screening candidate compounds for treating seizure disorders.

A. Generation of an Antibody Specific for the MAPK Phosphorylation Site in Kv4.2 (MAPK-Kv4.2 Phospho-selective Antibodies)

We have identified three ERK phosphorylation sites within the carboxy terminal cytoplasmic domain of Kv4.2 (Ser602, Ser607, and Thr616) and developed a phospho-selective antibody that recognize Kv4.2 when phosphorylated at all three sites. Phosphoselective antibody was generated in animals immunized with a synthetic peptide corresponding to the sequence in Kv4.2 which is phosphorylated by MAPK. Briefly, a synthetic peptide comprising 24 amino acids shown in single letter amino acid code with the N-terminus (NH3) and C-terminus (COOH) indicated (the “·” above an amino acid denotes phosphorylation) was synthesized by automated peptide synthesis.

| | |
|---|---|
| NH3- I S I P T P P V T T P E G D D R P E S P E Y S C - COOH | (SEQ ID NO:1) |

This sequence corresponds to the sequence in Kv4.2 phosphorylated by MAPK.

Because of the small size of the peptide (antigen) it does not stimulate an immunogenic response. To render it fully immunogenic, the peptide was conjugated to the carrier protein, KLH, a protein with known immunogenicity. Maleimide-activated KLH was conjugated to the peptide under standard conditions, with the conjugation occurring through the cystine sulfhydryl group at the carboxy-terminus of the peptide and the activated (sulfhydryl-reactive) maleimide of KLH. The peptide also was similarly coupled to ovalbumin for western blots to screen the antisera.

The KLH coupled peptide was sent to Cocalico Biologicals, Inc. (Reamstown, PA) for antibody generation using a standard protocol. Antiserum was obtained and used diluted or the antibodies were affinity purified by affinity chromatography versus Kv4.2 peptide bound to a carrier resin (e.g. sepharose).

Phosphorylated and unphosphorylated carboxy-terminal Kv4.2 protein fused to glutathionine S-transferase (GST) was also used to test antibody specificity. The carboxy-terminal fusion protein was made as follows: First, the carboxy-terminal cytoplasmic domain of Kv4.2 representing residues at positions 411 to 630 of mature Kv4.2 (see residues 1-630, shown in Isbrandt et al. , Genomics 64(2):144-154 (2000); see also GenBank accession no. AAF65618: http://www.ncbi.nlm.nih.gov:80/entrez/guery.fcgi?cmd=Retrieve&db=Protein&list_uids=7648673&dopt=GenPept) was spliced C-terminal to the sequence encoding glutathionine S-transferase in the expression vector pGEX-KN (Clontech, Palo Alto, Calif.). Several candidate consensus sequences for phosphorylation by MAPK were located in Kv4.2 carboxy terminal fragment. It then was determined that the recombinant GST-fusion protein construct of the carboxy-terminal cytoplasmic domain of Kv4.2 representing residues 411 to 630 was an effective substrate for MAPK phosphorylation in vitro as indicated by labelling of purified MAPK in the presence of ³²P-ATP and Mg⁺⁺. Phosphopeptide mapping and automated amino acid sequencing were used to determine that residues Thr602, Thr 607, and Ser 616 within the Kv4.2 sequence are phosphorylated by MAPK using the carboxy-terminal GST fusion construct.

The phospho-selectivity of antibodies raised against the phosphorylated 24 residue Kv4.2 peptide was evaluated by Western blotting against the phosphorylated oval-coupled synthetic peptide and the phosphorylated and unphosphorylated carboxy-terminus GST-fusion protein. The Western blots demonstrated immunoreactivity with the synthetic phospho-peptide coupled to ovalbumin and selective immunoreactivity with the phosphorylated GST-fusion protein corresponding to the Kv4.2 carboxy-terminus. The immunoreactivity was blocked by pre-incubation of the antibodies with the synthetic phospho-peptide. The antibodies failed to react with the unphosphorylated GST-fusion protein corresponding to the Kv4.2 carboxy-terminus. This shows that the antibody can specifically detect phosphorylated Kv4.2 MAPK sites and is useful to detect MAPK generated phosphorylation of Kv4.2 in tissues.

B. Input-specific Changes in Phospho-Kv4.2 in Hippocampus of Normal Animals

Rat hippocampal slices were bathed in vehicle with MEK inhibitor U0126 (20 μM) or vehicle alone (CTL). CA1 was dissected and Western blotted using MAPK-Kv4.2 phospho-selective antibodies. The Western blots showed a basal phosphorylation of Kv4.2 by MAPK in the hippocampus which is reduced in the presence of the MEK inhibitor, U0126 (inhibitor decreased detection by MAPK-Kv4.2 phospho-selective antibody 61.14%±8.74% of control, n=15; ***p<0.0001, unpaired t-test). These results indicate that phosphorylation and inhibition of Kv4.2 potassium channels is accomplished by MAPK.

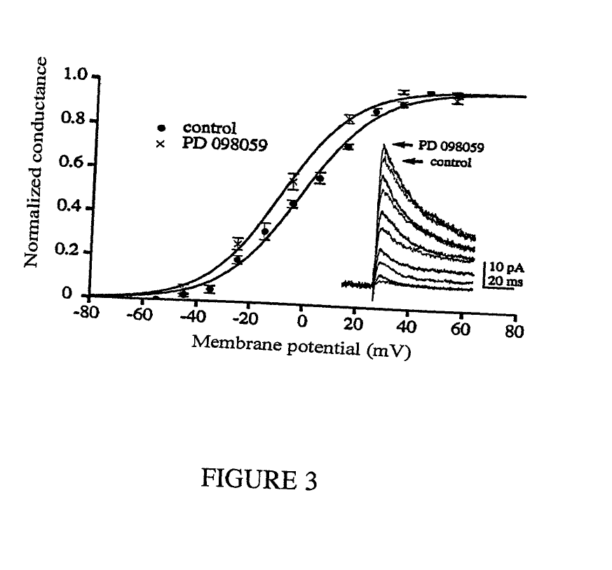

The functional consequence of MAPK inhibition on transient K⁺currents composed of Kv4.2 subunits as recorded in distal dendrites of hippocampal area CA1 was evaluated. The MEK inhibitor PD098059 (50 M) or vehicle was applied to the hippocampal CA1 slices at least 20 min prior to recording conductance. Conductance with inhibitor was increased compared to controls at almost all voltages applied, resulting in a phase shift of the inhibitor to the left of the control (V_{½}=−9mV, n=7) before compound (V_{½}=−2mV, n=10, p<0.0005) after compound. A single hippocampal slice treated first with vehicle followed by a wash and subsequent treatment with inhibitor showed less transient current at all potentials in the physiologic range with vehicle alone compared to the inhibitor (FIG. 3). Similar results were obtained using the MEK inhibitor U0126 (Control 85.55% and SL327 61.83% of control, n=2).

A hyperpolarizing shift in the voltage-dependence of K⁺currents, recorded in dendrites following a reduction in the levels of activated MAPK, indicates that MAPK activation causes a net increase in hippocampal excitability. Because the K⁺channel species expressing a transient current in hippocampal pyramidal neurons appears to be Kv4.2 channels, these results showing that MAPK causes hippocampal neural excitability indicates that inhibiting MAPK or the MAPK cascade provides a means to reduce or inhibit excessive neural excitability or reduce seizures in a seizure disorder.

C. Immunohistochemical Detection of Hippocampal Cells With MAPK-Following Kainate Administration

[insert*: see symbol after “antibody”] Immunohistochemistry using phospho-Kv4.2 antibody (phospho-Kv4.2) to the ERK sites was performed as described in Example 1 for the same animal groups. Representative transverse hippocampal sections labeled with the phospho-Kv4.2 antibody demonstrate that, after kainate-induced seizures (kainate), there is an increase in phospho-Kv4.2 in areas that have some basal levels of staining in the control. These areas include stratum oriens and stratum radiatum of areas CA1 and CA3 and the dentate hilus. Notably, there is also labeling of the CA3 pyramidal cell body layer following seizures that was not seen in the control condition. so=stratum oriens, sp=stratum pyramidale, sr=stratum radiatum, slm=stratum lacunosum moleculare, ml−dl=molecular layer of dentate gyrus, gc-dg=granule cells of the dentate gyrus.

Immunohistochemistry using the antibodies to the phosphorylation sites for MAPK and PKA on Kv4.2 and with the total and phospho-MAPK antibodies in control animals showed a high degree of correlation in the subcellular distribution of MAPK phosphorylated Kv4.2. These results show that Kv4.2 is a useful target to inhibit the downstream effects of MAPK activation in the hippocampus in vivo.

D. Detection of Kv4.2 phosphorylation in the hippocampus following SL327 pretreatment of control and kainate-treated animals

Western blotting using the phospho-Kv4.2 antibody to the ERK sites was performed on membrane fractions from kainate and control animals pretreated with SL327 as described in Example 1. See also Adams et al., J. Neurochem. 75:2277-2287 (2000), and Anderson et al., J. Biol. Chem. 275:5337-5346 (2000). Animals were sacrificed 30 minutes after kainate (before overt seizures) and 1 hour after continuous motor seizures.

A significant increase in phospho-Kv4.2 in hippocampus was detected following 1 hr of kainate-induced limbic motor seizures [KA+Vehicle: CA1 535.5±84.1, CA3 442.0±57.0, and dentate 1177.0±361.1% of CTL+Vehicle (KA+Vehicle n=14, CTL+Vehicle n=16)(p<0.0001)]. This effect was attenuated by pretreatment with 400 mg/kg SL327 [KA+SL327: CA1 152.8±41.4, CA3 170.8±16.6, and dentate 66.6±10.36% of CTL+SL327 (KA+SL327 n=5, CTL+SL327 n=7)(p=ns)]. These findings identify Kv4.2 as a candidate effector of ERK in seizures and opens up the possibility of ERK regulation of hippocampal excitability in this context through modulation of K⁺channel activity.

Interestingly, at the early time point (30 min after kainate injection) when there is already increased ERK activation but no apparent seizures, ERK phosphorylation of Kv4.2 was not significantly increased above control levels [kainate: CA1 48.80±6.95, CA3 128.70±29.12, dentate 109.10±11.09% control (CA1 p<0.001, CA3 and dentate ns)(n=6)]. The observations of ERK activation imply that ERK phosphorylation of Kv4.2 is slower than the kinetics of ERK activation itself.

### EXAMPLE 5

### CREB as a Downstream MAPK-cascade Target in Seizure Disorders

CREB activation is deemed a critical molecular target of the second messenger pathways which are activated in various forms of synaptic plasticity. Activated CREB binds to a specific DNA sequence, the cyclic AMP response element (CRE), and initiates transcription of the downstream gene.

Phosphorylation at Serl₃₃ is necessary to activate CREB, and this site is a substrate for PKA and Calcium/calmodulin-dependent protein kinases. CREB activation is coupled to the MAPK cascade via the intervening kinase RSK and activation of PKA and PKC lead to MAPK-dependent phosphorylation of CREB in area CA1 of hippocampus. Whether CREB activation plays a role in the long-term sequelae of recurrent seizure activity as in an acute episode of status epilepticus was evaluated.

A. Kainate-induced Status Epilepticus is Associated With Modulation of CREB Phosphorylation in Hippocampus

Western blots prepared using CA1, CA3 and dentate regions of the hippocampus from rats following kainate-induced status epilepticus (KA, n=3) or vehicle alone (CTL, n=3) were performed using phospho-CREB antibodies. There is an increased phosphorylation of CREB for CA1 and dentate regions (CA1 162.9±29.4% and dentate 138.0±4.4% of control). There is a small but significant decrease in CREB phosphorylation in CA3 (CA3 80.4±9.0%; *p<0.05, ***p<0.001). The results show that kainate-induced status epilepticus is associated with increased CREB phosphorylation in the CA1 and dentate regions of the hippocampus and that CREB phosphorylation decreases. Overall these data indicate dynamic regulation of CREB activation in status epilepticus.

B. CREB Activation in CA1 is Reduced by MAPK Cascade Inhibitors

Hippocampal region CA1 was removed 2 hours after rats were treated IP with the MEK inhibitor, SL327 (100 mg/kg) or vehicle alone. Western blots were developed using the phosho-CREB antibody. The results show that SL327 reduces the amount of activated CREB in the CA1 region of the hippocampus. These findings demonstrate the potential for using MAPK cascade inhibitors to reduce the amount of active CREB in hippocampal area CA1 and, therefore, reduce or inhibit excessive neural excitability or seizures consequent to CREB activation.

### EXAMPLE 6

### Vinylogous Cyanamides as MAPK Activation Inhibitors

Vinylogous cyanamides, useful in the methods disclosed here, can be prepared in a number of ways known to one skilled in the art of organic synthesis. Preferred methods include, but are not limited to, those described below. The reactions are performed in a solvent appropriate to the reagents and materials employed and suitable for the transformations being effected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a compound having MAPK inhibitory activity and, optionally, the ability to cross the blood-brain barrier. Another major consideration in the planning of any synthetic route in this field is the judicious choice of the protecting group used for protection of the reactive functional groups present in the compounds described in this invention. An authoritative account describing the many alternatives to the trained practitioner is Greene and Wuts, PROTECTIVE GROUPS IN ORGANIC SYNTHESIS (Wiley and Sons, 1991).

Abbreviations used in the Examples are defined as follows: “1×” for once, “2×” for twice, “3×” for thrice, “° C.” for degrees Celsius, “eq” for equivalent or equivalents, “g” for gram or grams, “mg” for milligram or milligrams, “mL” for milliliter or milliliters, “¹H” for proton, “h” for hour or hours, “M” for molar, “min” for minute or minutes, “MHz” for megahertz, “MS” for mass spectroscopy, “NMR” for nuclear magnetic resonance spectroscopy, “rt” for room temperature, “tlc” for thin layer chromatography, “v/v” for volume to volume ratio. “α”,“β”, “R” and “S” are stereochemical designations familiar to those skilled in the art.

A. The General Class of Vinylogous Cyanamides (Formula 1a and 1b)

For use according to the present invention, a class of vinylogous cyanamides is suitable for inhibiting phosphorylation of MAPK and its activation. The class encompasses compounds of formula Ia and Ib:

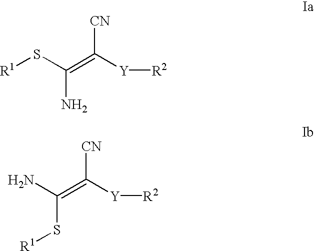

as well as stereoisomers and pharmaceutically acceptable salt form thereof, wherein:

R¹ is phenyl, naphthyl, 2,3-dihydroindol-5-yl or a 5-6 membered heteroaryl ring with 1-4 heteroatoms selected from N, NH, O, and S, and R¹ is substituted with 0-2 R^{a};

R^{a} is selected from H, C1, F, Br, l, C₁₋₄ alkyl, C₁₋₄ alkoxy, OH, CH₂OH, NH₂, (C₁₋₃ alkyl)NH, (C₁₋₃ alkyl)₂N, (H₂NCH₂C(O))NH, (H₂NCH(CH₃)C(O))NH, (CH₃NHCH₂C(O))NH, ((CH₃)₂NCH₂C(O))NH, CF₃, OCF₃, —CN, NO₂, C(O) NH₂, and CH₃C(O)NH;

Y is selected from phenyl substituted with 0-5 R^{b}, naphthyl substituted with 0-5 R^{b}, and CHR³;

R^{b} is selected from H, Cl, F, Br, l, C₁₋₄ alkyl, OH, C₁₋₄ alkoxy, CH₂OH, CH(OH)CH₃, CF₃, OCF₃, —CN, NO₂, NH₂, (C₁₋₃ alkyl) NH, (C₁₋₃ alkyl)₂N, and C(O)O—C₁₋₄ alkoxy;

R² is selected from H, R^{2a}, C(O)R^{2a}, CH(OH)R^{2a}, CH₂R^{2a}, OR^{2a}, SR^{2a}, and NHR^{2a};

R^{2a} is selected from phenyl, naphthyl, and a 5-6 membered heteroaryl ring with 1-4 heteroatoms selected from N, NH, O, and S, and R^{2a} is substituted with 0-5 R^{b};

R³ is phenyl substituted with 0-2 R^{c} or naphthyl substituted with 0-2 R^{c}; and,

R^{c} is selected from H, Cl, F, Br, l, C₁₋₄ alkyl, OH, C₁₋₄ alkoxy, CH₂OH, CH(OH)CH₃, CF₃, OCF₃, —CN, NO₂, NH₂, (C₁₋₃ alkyl)NH, (C₁₋₃ alkyl)₂N, and C(O)O—C₁₋₄ alkoxy.

B. Synthesis of the General Class of Vinylogous Cyanamides (Formula 3)

Vinylogous cyanamides (3) may be synthesized by the route described in Scheme 1. A thiol 1, such as a thiophenol, may be treated with a malononitrile such as malononitrile 2 in the presence of a base catalyst such as triethylamine, DBU, Hunig's base, or aqueous base (for example, 10% NaOH), etc., in a nonreactive solvent such as THF, acetone, etc., to yield the vinylogous cyanamide 3. The reaction medium can be degassed to eliminate the presence of oxygen which can facilitate disulfide formation via the dimerization of thiol 1. The vinylogous cyanamide is frequently isolated as a mixture of Z- and E-isomers and the melting point varies significantly with isomer composition. A crystalline single isomer or material enriched in one isomer may sometimes be obtained by spontaneous crystallization of one isomer, recrystallization, or stirring solid in a solvent which dissolves only part of the material. Alternatively, isomers may sometimes be separated by chromatography. However, the double bond in 3 isomerizes very easily. NMR spectroscopy of a single isomer in DMSO-d₆ shows that an equilibrium mixture of Z- and E-isomers is generated faster than the spectrum could be obtained (about 5 minutes). Isomerization also takes place in other solvents such as water, acetone, methanol, and chloroform, but more slowly than in DMSO. Rapid NMR in one of these solvents may be used to establish isomeric composition. For in vitro assays, the compounds may be dissolved in DMSO to ensure that an equilibrium mixture of isomers is tested.

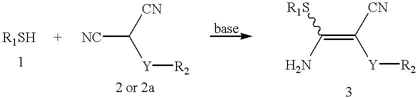

Many thiols (1) are commercially available. Alternatively, there are many methods for their synthesis familiar to one skilled in the art. For example, aryl or heterocyclic anions may be quenched with sulfur to yield thiols. Chem. Pharm. Bull. 37(1):36 (1989). Displacement of aryldiazonium salts with EtOCS₂K leads to aryl thiols. Collect. Czech. Chem. Commun. 55:1266 (1990). The Newman rearrangement of phenols via their dimethylthiocarbamates leads to thiophenols. ORGANIC SYNTHESES VI, page 824(1988).

When the Y group in Scheme 1 is substituted phenyl or naphthyl, the malononitrile precursors (2) to the compounds of this invention may be prepared by one of the three routes shown in Scheme 2. In the first route, aryl iodides 4 may be treated with malononitrile in the presence of a copper catalyst to yield arylmalononitriles 2. J. Org. Chem. 58:7606-7 (1993). Malononitrile can also be coupled to aryl halides 4 (X=halide) using (Ph₃P)₂PdC1₂ or Pd(Ph₃P)₄ in THF. J. Chem. Soc. Chem. Comm. 932-3 (1984). The aryl iodides needed for these methods are commercially available or prepared by methods familiar to one skilled in the art. In particular, aryl iodides may be prepared by iodination with a source of electrophilic iodine, such as iodine monochloride, or by diazotization of anilines.

Scheme 2: Preparation of malononitriles 2 When Y is a Substituted Phenyl or Naphthyl

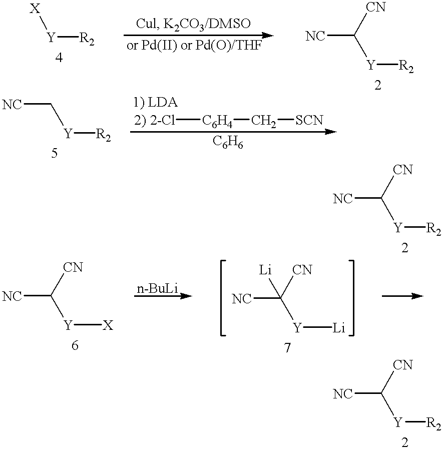

Arylmalononitriles also can be prepared from aryl acetonitriles as shown the second route in Scheme 2. Aryl acetonitriles 5 may be deprotonated with a base, such as LDA, and quenched with a electrophilic source of cyanide, such as cyanogen chloride (J. Org. Chem. 21:919 (1 966)) or 2-chlorobenzylthiocyanate (J. Org. Chem. 48:2774-5 (1983)) to yield malononitrile 2. Along the same lines, acetonitrile 5 also be acylated in the presence of NaOMe with dimethyl carbonate to form the methyl cyanoacetate (not shown in Scheme 2). Conversion of the methyl ester to a nitrile group via procedures familiar to one skilled in the art leads to malononitrile 2 (J. Am. Chem. Soc. 32:119 (1904)). The aryl acetonitriles needed for these methods are commercially available or prepared by methods familiar to one skilled in the art, for example, from aryl acetamides or from toluenes. When R₂ is an optionally substituted phenoxy group, the initial step in the preparation of the compounds of this invention may be an Ullmann condensation between an aryl halide and a phenol. (For useful protocols, see U.S. Pat. No. 4,288,386 and Tetrahedron 15:144-153(1961).) A methyl substituent on either of these substrates may be subsequently converted to a —CH2CN group by free radical halogenation, with a reagent such as N-bromosuccinimide, followed by displacement with cyanide.

As seen in the third route shown in Scheme 2, arylmalononitriles 2 may also be synthesized from simpler bromo- or iodoarylmalononitriles. These bromo- or iodo-substituted arylmalononitriles may be prepared by either of the first two routes indicated in Scheme 2 for the preparation of malononitriles. Bromo- or iodo-substituted arylmalononitriles undergo halogen-metal exchange in the presence of two or more equivalents of an alkyllithium reagent, such as n-butyllithium, to form dianion intermediate 7. This process may be carried out in an ethereal solvent such as THF at a temperature of −78 to 0° C. The dianion may be quenched in situ with one equivalent of an electrophile, such as an aldehyde, alkyl halide, disulfide, ester, or ketone, to yield a substituted malononitrile 2 with a new R₂ group attached to the former site of the bromine or iodine atom. This process is illustrated in more detail in Scheme 3 for the case where Y is a 1,3-disubstituted phenyl group. 3-Bromophenylmalononitrile (6) may be converted to dianion 7a

Scheme 3: An Illustration of the Use of Arylmalononitrile Dianions

[chemical structure]

by deprotonation and halogen-metal exchange with 2 equivalents of n-butyllithium in THF at −78° C. The dianion may be treated in situ with an aldehyde to produce hydroxy-phenylmalononitriles 8. Hydroxy-phenylmalononitriles 8 may be oxidized to the corresponding keto-phenylmalononitrile 9 using MnO₂ or a variety of other oxidizing agents familiar to one skilled in the art. Compounds 8 and 9 may be reduced to the corresponding CH₂R₂-substituted phenylmalononitriles 10 using hydrogen and a noble metal catalyst, NaBH₄ and TFA (Synthesis 763-5 (1 978)), or other procedures familiar to one skilled in the art. Malononitriles 8, 9, and 10 may be treated with thiols 1 to yield the compounds of this invention. It must be noted that although only the meta-bromo isomer of 6 is pictured in Scheme 3, one trained in the art may apply this methodology using other aryl halides and electrophiles to prepare isomers and compounds with different Y groups.

Scheme 4: Preparation of Malononitriles When Y is CHR₃

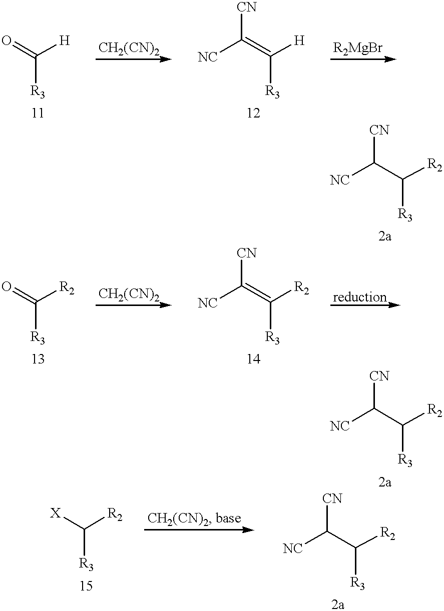

When Y is CHR₃, malononitrile precursors useful for preparation of the compounds of this invention have structure 2a and may be prepared as shown in Scheme 4. Knoevenagel condensation (Organic Reactions 15:204-209 (1967)) between an aldehyde 11 or a ketone 13 may be used to produce alkylidene malononitriles 12 or 14. Conjugate addition of a Grignard or organolithium reagent to 12 affords the malononitrile precursors 2 used in Scheme 1. Alternatively, alkylidene malononitriles 14 may be reduced to malononitriles 2a with sodium borohydride, catalytic hydrogenation or other reducing agents familiar to one skilled in the art. A third alternative is to alkylate malononitrile with an alkyl halide 15 (X=halide).

B. Synthesis of Z- and E-α-[amino[(4-aminophenyl)thio]methylene]-2-(trifluoromethyl)benzeneacetonitrile (SL 327)

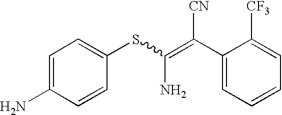

Part I. Preparation of 2-[(2-trifluoromethyl)phenyl]malononitrile

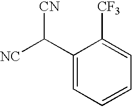

A mixture of 2-trifluoromethyl-1-iodobenzene (21.76 g, 0.08 mol, 1 eq), malonitrile (10.56 g, 0.16 mol, 2 eq), copper(l) iodide (1.52 g, 0.008 mol, 0.1 eq), potassium carbonate (11.04 g, 0.32 mol, 4 eq), and 200 mL DMSO was stirred and heated at 120° C. for 21 h. The reaction mixture was cooled and poured into 1.2 L of 0.5 M HCl. The mixture was filtered and extracted with ethyl acetate. The organic layer was dried (MgSO₄) and the solvent removed in vacuo to yield an oil. This oil was purified by flash chromatography on silica gel with 3:1 hexane/ethyl acetate to yield 4.46 g (27%) of 2-[(2-trifluoromethyl)phenyl] malononitrile as a yellow oil. ¹H-NMR (CDC1₃) δ:8.05-7.10 (m, 4H); 5.30 (s, 1H).

Part II. Preparation of α-[amino[(4-aminophenyl)thio]methylene]-2-(trifluoromethyl) benzeneacetonitrile

2-[(2-Trifluoromethyl)phenyl]malononitrile, the product form Part I (3.07 g, 14.6 mmol, 1.1 eq) was mixed with freshly distilled 4-aminothiophenol (1.66 g, 13.3 mmol, 1 eq), and THF (25 mL). The reaction flask then was degassed by placement under vacuum, followed by flushing with N₂ several times to prevent disulfide formation. After cooling to −78° C., triethylamine (1.85 mL, 13.3 mmol, 1 eq) was added via syringe and the flask degassed once more. The contents were allowed to warm to room temperature and the mixture was stirred overnight. TLC the following morning showed no malononitrile present, only thiol. Therefore, another 0.2 equivalents of malononitrile were added followed by degassing, followed by 0.5 equivalents of triethylamine, followed by degassing. TLC after a few hours no starting material was present. The reaction was worked up after stirring over the weekend at room temperature. The solvent was removed in vacuo and the residue was purified by flash chromatography on silica gel with 25-1 00% ethyl acetate in hexane. Two fractions were isolated. The faster eluting fraction yielded 1.63 g of a tan oily solid. The slower eluting fraction yielded 2.61 g of a tan oily solid. Both compounds were recrystallized from n-butylchloride. The faster eluting compound yielded 274 mg of a white solid (m.p. 147.0-148.0° C.). This compound proved to be the E isomer of the titled compound through NMR NOE experiments. The slower eluting compound yielded 1.85 g of a white solid (m.p. 130.0-130.5° C.). This compound proved to be the Z isomer of the titled compound through NMR NOE experiments. Anal. calcd. for C₁₆H₁₂F₃N₃S (faster eluting isomer): C, 57.31; H, 3.62; F, 17.00; N, 12.53; S, 9.56. Found: C, 57.19; H, 3.75; F, 16.83; N, 12.24; S, 9.50. Anal. calcd. for C₁₆H₁₂F₃N₃S (slower eluting isomer): C, 57.31; H, 3.62; F, 17.00; N, 12.53; S, 9.56. Found: C, 57.28; H, 3.80; F, 16.96; N, 12.37; S, 9.22. ¹H-NMR (faster eluting isomer) (CDC1₃) δ 7.75 (d, 1H, J=7 Hz); 7.57 (t, 1 H, J=7 Hz); 7.49 (t, 1H, J=7 Hz); 7.47 (d, 1H, J=7 Hz); 7.24 (d, 2H, J=7 Hz); 6.66 (d, 2H, J=7 Hz). ¹H-NMR (slower eluting isomer) (CDC1₃) δ 7.75 (d, 1H, J=7 Hz); 7.58 (t, 1 H, J=7 Hz); 7.48 (t, 1H, J=7 Hz); 7.43 (d, 1H, J=7 Hz); 7.40 (d, 2H, J=7 Hz); 6.68 (d, 2H, J=7 Hz).

The invention thus has been disclosed broadly and illustrated in reference to representative embodiments described above. Those skilled in the art will recognize that various modifications can be made to the present invention without departing from the spirit and scope thereof. All publications, patent applications and issued patents, are herein incorporated by reference to the same extent as if each individual publication, patent application or issued patent were specifically and individually indicated to be incorporated by reference in its entirety.

What is claimed is:

1. A method of reducing or inhibiting excessive neuronal excitability in an individual resulting from an increased mitogen-activated protein kinase (MAPK) activity in neurons of the individual, comprising administering an effective amount of a compound that reduces the amount of MAPK activity in the neurons.

2. The method of claim 1 wherein the excessive neuronal excitability results in a seizure disorder.

3. The method of claim 2, wherein the seizure disorder is epilepsy.

4. The method of claim 1, wherein the compound inhibits phosphorylation of MAPK.

5. The method of claim 1, wherein the compound is a vinylogous cyanamide of the formula I(a) or I(b)
or stereoisomer or pharmaceutically acceptable salt form thereof, wherein; 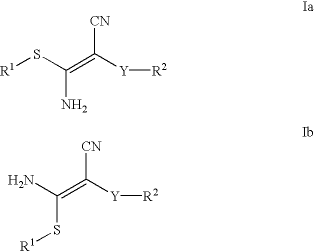
R¹ is phenyl, naphthyl, 2,3-dihydroindol-5-yl or a 5-6 membered heteroaryl ring with 1-4 heteroatoms selected from N, NH, O, and S, and
R¹ is substituted with 0-2 R^{a};
R^{a} is selected from H, C1, F, Br, I, C₁₋₄ alkyl, C₁₋₄ alkoxy, OH, CH₂OH, NH₂, (C₁₋₃ alkyl)NH, (C₁₋₃ alkyl)₂N, (H₂NCH₂C(O))NH, (H₂NCH(CH₃)C(O))NH, (CH₃NHCH₂C(O))NH, ((CH₃)₂NCH₂C(O))NH, CF₃, OCF₃, —CN, NO₂, C(O) NH₂, and CH₃C(O)NH;
Y is selected from phenyl substituted with 0-5 R^{b}, naphthyl substituted with 0-5 R^{b}, and CHR³;
R^{b} is selected from H, Cl, F, Br, I, C₁₋₄ alkyl, OH, C₁₋₄ alkoxy, CH₂OH, CH(OH)CH₃, CF₃, OCF₃, —CN, NO₂, NH₂, (C₁₋₃ alkyl) NH, (C₁₋₃ alkyl)₂N, and C(O)O—C₁₋₄ alkoxy;
R² is selected from H, R^{2a}, C(O)R^{2a}, CH(OH)R^{2a}, CH₂R^{2a}, OR^{2a}, SR^{2a}, and NHR^{2a};
R^{2a} is selected from phenyl, naphthyl, and a 5-6 membered heteroaryl ring with 1-4 heteroatoms selected from N, NH, O, and S, and R^{2a} is substituted with 0-5 R^{b};
R³ is phenyl substituted with 0-2 R^{C} or naphthyl substituted with 0-2 R^{C}; and, R^{C} is selected from H, Cl, F, Br, I, C₁₋₄ alkyl, OH, C₁₋₄ alkoxy, CH₂OH, CH(OH)CH₃, CF₃, OCF₃, —CN, NO₂, NH₂, (C₁₋₃ alkyl)NH, (C₁₋₃ alkyl)₂N, and C(O)O—C₁₋₄ alkoxy.

6. The method of claim 5, wherein the compound is of formula II 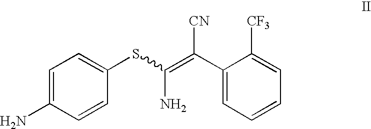
and wherein the compound includes the Z- and E- isomers thereof.

7. The method of claim 1, wherein administering the compound reduces the amount of MAPK activity in the neuron by inhibiting phosphorylation or kinase activity in the MAPK cascade upstream of MAPK.

8. The method of claim 7, wherein the upstream kinase is selected from the group consisting of MEK, Raf-1, Ras, B-Raf and Rap1.

9. The method of claim 1, wherein the MAPK is an extracellular signal-regulated kinase (ERK).

10. The method of claim 9, wherein the ERK is ERK1 or ERK2.

11. A method of reducing or inhibiting excessive neuronal excitability in an individual resulting from an increased mitogen-activated protein kinase (MAPK) activity in neurons of the individual, comprising administering an effective amount of a compound that inhibits phosphorylation or kinase activity of the MAPK cascade downstream of MAPK.

12. The method of claim 11, wherein the compound inhibits phosphorylation or kinase activity of a transcriptional factor.

13. The method of claim 11, wherein the transcriptional factor is CREB.

14. The method of claim 12, wherein the compound inhibits phosphorylation of potassium channels.

15. The method of claim 14, wherein the potassium channels are Kv4.2 potassium channels.

16. The method of claim 11, wherein the excessive neuronal excitability results in a seizure disorder.

17. The method of claim 16, wherein the seizure disorder is epilepsy.

18. A method of reducing or inhibiting seizures in an individual suffering from a seizure disorder, resulting from increased mitogen-activated protein kinase (MAPK) activity, comprising administering an effective amount of a compound that inhibits phosphorylation of potassium channels.

19. The method of claim 18, wherein the potassium channels are Kv4.2 potassium channels.

20. The method of claim 18, wherein the seizure disorder is epilepsy.

21. The method of claim 18, wherein phosphorylation of potassium channels are inhibited by inhibiting phosphorylation of MAPK.
